(19) United States
(12) Patent Application Publication
(10) Pub. No.: US 2018/0092870 A1
(43) Pub. Date: Apr. 5, 2018

(54) Butyrate Based Skin Formulation

(71) Applicants: Francis M. Lobo, New Haven, CT (US); Christina Price, New Haven, CT (US)

(72) Inventors: Francis M. Lobo, New Haven, CT (US); Christina Price, New Haven, CT (US)

(21) Appl. No.: 15/725,004

(22) Filed: Oct. 4, 2017

Related U.S. Application Data
(60) Provisional application No. 62/403,874, filed on Oct. 4, 2016.

(51) Int. Cl. A61K 31/19; A61K 9/00; A61K 9/06

(52) U.S. Cl. CPC A61K 31/19; A61K 9/06; A61K 9/0014

(57) ABSTRACT

A topical composition for the treatment of inflammatory skin conditions including a butyrate active ingredient and a dermatologically acceptable carrier. The compositions include about 0.01 w/w % to about 20 w/w % butyrate, preferably a butyrate metal salt, and are free of a steroid. The compositions can be to the skin of a patient in need thereof in a method of treating inflammatory conditions of the skin.

### FIELD OF THE INVENTION

The present invention relates to pharmaceutical compositions comprising butyrate and methods of use thereof for the prevention and treatment of topical inflammation conditions.

### BACKGROUND OF THE INVENTION

Conventional anti-inflammatory skin products contain steroids. For example, Cortizone 10 Hydrocortisone gel, cream, or ointment is widely used as an over-counter product for the temporary relief of various inflammation conditions, such as minor skin irritations, itching, and rashes caused by eczema, insect bites, poison ivy, poison oak, poison sumac, soaps, detergents, cosmetics, and jewelry. However, the active ingredient, hydrocortisone, like all other steroids, can weaken a person's immune system, which could worsen an existing infection or make the person more susceptible to new infections. When applied topically to the skin for long periods of time hydrocortisone can cause skin thinning and fragility as well as pigment changes.

Topical, anti-inflammatory compositions which make use of non-steroidal agents have been disclosed. For example, U.S. Pat. No. 4,360,518 to Rovee et al. discloses a pharmaceutical composition for topical treatment of skin inflammation comprising an anti-inflammatory corticosteroid and a non-steroidal anti-inflammatory agent. However, a disadvantage of this combination is that it still utilizes a steroidal drug, which often exhibits undesired local and systemic side effects when used for prolonged periods. Additionally, many of the steroidal drugs must be used systemically as opposed to topically.

U.S. Pat. No. 7,229,650 to Callaghan et al. discloses a method of treating and preventing inflammatory disorders via a topical composition containing an effective amount of feverfew extract. Although this disclosure eliminates the negative side effects that come with steroidal drug use, it introduces its own set of adverse side effects. First, feverfew is known to have adverse interactions when taken with some other medications. Namely, feverfew may decrease how quickly the liver breaks down certain medications. Additionally, taking feverfew along with some medications that are broken down by the liver can increase the side effects associated with those medications. Lastly, feverfew has the potential to slow blood clotting, so when taken together with medications that also slow blood clotting, there is an increased risk of bruising and bleeding.

There is a need for improved steroid free, topical anti-inflammatory compositions.

There is a need for steroid free, topical anti-inflammatory compositions that avoid the adverse side effects associated with other topical, anti-inflammatory compositions.

### SUMMARY OF THE INVENTION

The foregoing objectives are achieved by provision of topical compositions for the treatment of inflammatory skin conditions comprising a butyrate active ingredient and a dermatologically acceptable carrier. The compositions are free of a steroid.

In some embodiments, the butyrate active ingredient is a butyric acid, a butyrate salt or a hydrate thereof. In certain of those embodiments, the butyrate active ingredient is a metal butyrate salt. In certain preferred embodiments, the butyrate active ingredient is selected from the group consisting of sodium butyrate, potassium butyrate, lithium butyrate and mixtures thereof.

In certain embodiments, the composition comprises about 0.01 w/w % to about 20 w/w % butyrate. In certain of those embodiments, the composition comprises about 0.05 w/w % to about 10.0 w/w % butyrate. In some preferred embodiments, the composition comprises about 0.1 w/w % to about 5.0 w/w % of butyrate.

In preferred embodiments, the composition is free of a feverfew extract.

In some embodiments, the composition further comprises a fragrance. In some of those embodiments, the fragrance is a natural oil. In certain of those embodiments, the oil is a grapefruit-based oil.

In preferred embodiments, the composition is a lotion or cream.

In certain preferred embodiments, a topical composition for the treatment of inflammatory skin conditions comprises about 0.1 w/w % of a butyrate metal salt and a dermatologically acceptable carrier, wherein the composition is free of a steroid. In especially preferred embodiments, the butyrate metal salt is sodium butyrate.

Also provided is a method of treating inflammatory conditions of the skin comprising administering the disclosed topical compositions to the skin of a patient in need thereof.

Further provided is a method of making topical butyrate compositions for treatment of inflammatory skin conditions comprising the steps of premixing a first mixture of water and thickener until it becomes homogeneous, adding a second mixture comprising moisturizer and conditioner to the first mixture and mixing until the two mixtures become homogeneous, premixing a third mixture comprising emollient, moisturizer, emulsifier, conditioner, skin barrier and preservative until it becomes homogeneous, heating the third mixture and adding it to the first and second mixtures to form a fourth mixture, mixing in a pH-neutralizer to the fourth mixture and letting the mixture cool; and adding the butyrate active ingredient and fragrance to the fourth mixture. In certain embodiments, the heating is to a temperature of about 75° C. In some embodiments, the cooling is to a temperature of about 50° C.

### DETAILED DESCRIPTION OF THE INVENTION

In one aspect, the present invention provides a pharmaceutical composition comprising butyrate as an active ingredient and a dermatologically acceptable carrier, which can be topically applied to an affected skin area for the treatment and prevention of skin inflammation.

The present invention addresses the legitimate steroid phobia of patients and physicians by providing a steroid-free composition (e.g., in the form of a body lotion) containing butyrate, which act as an anti-inflammatory agent. Butyrate (also known as butanoate) is the traditional name for the conjugate base of butyric acid (also known as butanoic acid). The formula of the butyrate ion is C₄H₇O₂⁻. Butyrate is a short chain fatty acid that is the natural fermentation product of many intestinal bacteria. Butyrate has been only used as a passive ionic component of a salt of another active ingredient (for example, hydrocortisone butyrate) in pharmaceutical compositions. In the present invention, the butyrate itself, for example, a plain butyrate salt wherein the counterpart of butyrate has no known anti-inflammation effect, is used as the active ingredient to control inflammation in the skin. It has been discovered that the butyrate itself possesses both preventive and therapeutic potential to counteract topical inflammation.

The active ingredient, butyrate, may be in the form of a butyrate salt wherein the counterpart of butyrate has no known anti-inflammation effect, and a hydrate of a butyrate salt. For example, the butyrate salt may be a metal butyrate salt wherein the counterpart of the salt is a metal ion. The butyrate salt may also have an organic counterpart which provides a positive charge to balance the negative charge of butyrate base. In preferred embodiments, the active ingredient is a metal butyrate salt. The metal butyrate salt may include, but is not limited to, sodium butyrate, potassium butyrate, and lithium butyrate. In some embodiments, the active ingredient may be a butyric acid.

The amount of the butyrate necessary to bring about the therapeutic treatment of skin inflammation is not fixed per se, and necessarily is dependent upon the severity and extent of the disease, the form of the butyrate employed, and the concentration of the butyrate when employed in association with a dermatologically acceptable carrier. The pharmaceutical composition may contain from about 0.01 w/w % to about 20.0 w/w %, preferably from about 0.05 w/w % to about 10.0 w/w %, and even more preferably from about 0.1 w/w % to about 5.0 w/w % of butyrate. In one embodiment, the quantity of butyrate is about 0.1 w/w %. In another embodiment, the quantity of butyrate is about 1.0 w/w %. By the term “w/w %”, it means a percentage of weight of butyrate (C₄H₇O₂⁻) over weight of the composition.

As used herein, the term “about” is defined as ±10%, preferably ±5%.

A suitable carrier should be one in which butyrate is soluble or is effectively solubilized (e.g., as an emulsion or microemulsion). Where employed, the carrier is inert in the sense of not bringing about a deactivation of the butyrate, and in the sense of not bringing about any adverse effect on the skin areas to which it is applied. A suitable carrier should be dermatologically acceptable and is compatible to other ingredients in the formulation. According to the present invention, suitable carriers include water, alcohols, oils and the like, chosen for their ability to dissolve or disperse butyrate and any other ingredients used in the formulation. The carrier may be in the form of a lotion, cream, ointment, soap, stick, or the like.

One issue of the butyrate formulation is that butyrate has an unpleasant foul smell. To cover or mask the smell, fragrance-masking natural oils are used. In some embodiments, citrus-based fragrance oils are used. In some embodiments, the fragrances oils are Sandal WoodShea (91-1026-26), Sweet Baby (91-1001-64-01), or a mixture thereof. In certain preferred embodiments, grapefruit-based oils are used. The amount of the fragrance oils may be from about 0.2 w/w % to about 2.5 w/w %.

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, thickening agents, skin barrier agents, etc., provided that they are physically and chemically compatible with other components of the composition.

Emollients include, but are not limited to, fatty esters, fatty alcohols, polyol (e.g., glycerin), mineral oils, polyether siloxane copolymers, docosahexanoic acid (DHA), meadowfoam seed oil, and the like.

Humectants include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, L-tyrosine, and the like.

Emulsifiers, which are also called emulsifying agents, include, but are not limited to, stearic acid, cetyl alcohol (as known as C-95 alcohol, or Crodacol™ C95, as sold by Croda Inc.), stearyl alcohol, steareth 2, steareth 20, acrylates/C10-30 alkyl acrylate cross polymers, silicones, dimethyl ethanolamine (DMAE), phosphatidylcholine (PPC), docosahexanoic acid (DHA), a blend of cetostearyl alcohol and polysorbate 60 (Polawax NF), and the like.

The conditioning agents include, but are not limited to, fatty acid (e.g., salicylic acid, ascorbic acid), fatty acid ester (e.g. myristyl myristate), and the like.

Chelating agents include, but are not limited to, ethylenediamine tetraacetic acid (EDTA) and derivatives and salts (e.g., sodium salt) thereof, dihydroxyethyl glycine, tartaric acid, and the like.

Antioxidants include, but are not limited to, butylated hydroxy toluene (BHT); vitamin C and/or vitamin C derivatives, such as fatty acid esters of ascorbic acid, particularly ascorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives (such as tocotrienol acetate, also known as vitamin E acetate); calcium pantothenates; green tea extracts; mixed polyphenols; and the like.

Preservatives include, but are not limited to, C₁-C₃ alkyl parabens, phenoxyenthanol (e.g., benzyl alcohol), capryl glycol/phenoxyethanol, and the like.

An exemplified thickening agent may be carbomer. An exemplified skin barrier agent may be meadowlactone.

The composition may also include one or more adjunctive active ingredients that are compatible to butyrate and are also beneficial to the skin.

Typical buffering agents are chemically and physically stable agents commonly found in cosmetics, and can include compounds that can also function as adjunct ingredients such as citric acid, malic acid, salicylic acid, and glycolic acid, and compounds that are basic, such as triethanolamine. Buffering agents are employed in many compositions. Preferably, the amount of buffering agent is one that results in compositions having a pH ranging from about 4.0 to about 8.5, more preferably from about 4.5 to about 7.0, even most preferably from about 5.0 to about 6.0. In one embodiments, the pH value of the composition is about 5.6, as measured at room temperature.

It should be noted that some ingredients listed above can function as just a tinting agent, an emollient, a skin conditioning agent, an emulsifying agent, a humectant, a preservative, an antioxidant, a perfume, a chelating agent, a thickening agent, or a skin barrier agent.

The exact mechanism as to how the butyrate formulation works to treat inflammatory skin conditions is unknown. Without wishing to be bound by theory, it is believed that, in theory, butyrate acts as an inhibitor of histone deacetylase, which allows for increased histone and other protein methylation. Among many genes influenced by histone methylation, and proteins stabilized by methylation, are the gene FOXP3 and its protein product FOXP3. FOXP3 is critical for the development of special immune regulating cells, T regulatory cells, which naturally control inflammation usually for the benefit of the body. The above theory is based on butyrate having been demonstrated to be a natural and dominant mechanism by which commensal bacteria help to control local inflammation.

Many preparations are known in the art, and include lotions containing oils and/or alcohols and emollients such as hydrocarbon oils and waxes, silicone oils, vegetable, animal or marine fats or oils, glyceride derivatives, fatty acids or fatty acid esters or alcohols or alcohol ethers, lecithin, lanolin and derivatives, polyhydric alcohols or esters, wax esters, sterols, phospholipids and the like, and/or generally also emulsifiers (nonionic, cationic or anionic), although some of the emollients inherently possess emulsifying properties. Other materials as well as processing techniques and the like are set forth in Remington's Pharmaceutical Sciences, 17th edition, 1985, Mack Publishing Company, Easton, Pa., which are incorporated by reference.

Examples of a composition and a method of preparing the composition in accordance with the present invention are illustrated here:

### Example 1—Formulation

| | | |
|---|---|---|
| A. | Water | 63.15% |
| | Thickener | 0.3% |
| B. | Skin Moisturizer | 5.0% |
| | Skin Conditioner | 0.1% |
| C. | Skin Emollient | 10.0% |
| | Skin Moisturizer | 10.0% |
| | Emulsifier | 5.0% |
| | Conditioner | 3.0% |
| | Skin Barrier | 1.0% |
| | Preservative | 1.0% |
| D. | PH-Neutralizer | 0.35% |
| | Sodium Butyrate | 0.1% |
| | Fragrance Oils | 1.0% |

### Example 2—Preparation

Step 1. Premix Phase A components until they become homogeneous;
Step 2. To the Phase A mixture add the components of Phase B and mix under they become homogeneous;
Step 3. Premix Phase C components until they become homogenous; heat to 75° C. and add to Phase C mixture to the mixture of Phase A and Phase B; then add pH-Neutralizer and mix;
Step 4. Cool the mixer from Step 3 to 50° C., add the rest of Phase D components and mix; further cool to room temperature; and
Step 5. Check specification of the mixture; transfer for filling and packaging.

The specification of the above skin cream is as follows:

| | |
|---|---|
| Physical Appearance | Thick Cream |
| Odor | Butyrate (suppressed by fragrances) |
| Viscosity | 78,000 |
| Color | White |
| pH | 5.6 at 23.9° C. |

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention.

What is claimed is:

1. A topical composition for the treatment of inflammatory skin conditions comprising a butyrate active ingredient and a dermatologically acceptable carrier, wherein the composition is substantially free of a steroid.

2. The topical composition of claim 1, wherein the butyrate active ingredient is a butyric acid, a butyrate salt, or hydrates thereof.

3. The topical composition of claim 2, wherein the butyrate active ingredient is a metal butyrate salt.

4. The topical composition of claim 3, wherein the butyrate active ingredient is sodium butyrate.

5. The topical composition of claim 3, wherein the butyrate active ingredient is potassium butyrate.

6. The topical composition of claim 3, wherein the butyrate active ingredient is lithium butyrate.

7. The composition of claim 1, wherein the composition comprises about 0.01 w/w % to about 20 w/w % butyrate.

8. The composition of claim 7, wherein the composition comprises about 0.05 w/w % to about 10.0 w/w % butyrate.

9. The composition of claim 8, wherein the composition comprises about 0.1 w/w % to about 5.0 w/w % of butyrate.

10. The composition of claim 1, wherein the composition is free of a feverfew extract.

11. The composition of claim 1, further comprising a fragrance.

12. The composition of claim 11, wherein the fragrance is a natural oil.

13. The composition of claim 12, wherein the oil is a grapefruit-based oil.

14. The composition of claim 1, wherein the composition is a lotion or cream.

15. A method of treating inflammatory conditions of the skin comprising administering a topical composition of claim 1 to the skin of a patient in need thereof.

16. A method of making the topical butyrate composition of claim 1 comprising the steps of:
premixing a first mixture of water and thickener until it becomes homogeneous;
adding a second mixture comprising moisturizer and conditioner to the first mixture and mixing until the two mixtures become homogeneous;
premixing a third mixture comprising emollient, moisturizer, emulsifier, conditioner, skin barrier and preservative until it becomes homogeneous;
heating the third mixture and adding it to the first and second mixtures to form a fourth mixture;
mixing in a pH-neutralizer to the fourth mixture and letting the mixture cool; and
adding the butyrate active ingredient and fragrance to the fourth mixture.

17. The method of claim 16, wherein the heating is to a temperature of about 75° C.

18. The method of claim 16, wherein the cooling is to a temperature of about 50° C.

19. A topical composition for the treatment of inflammatory skin conditions comprising about 0.1 w/w % of a butyrate metal salt and a dermatologically acceptable carrier, wherein the composition is free of a steroid.

20. The composition of claim 19, wherein the butyrate metal salt is sodium butyrate.
